(19) United States
(12) Patent Application Publication
(10) Pub. No.: US 2007/0093840 A1
(43) Pub. Date: Apr. 26, 2007

(54) Flexible shaft

(76) Inventors: Nicolas J. Pacelli, Culver, IN (US); Antony J. Lozier, Warsaw, IN (US); Sarah L. Thelen, North Manchester, IN (US); Billy N. Sisk, Claypool, IN (US)

Correspondence Address: ZIMMER TECHNOLOGY - BAKER & DANIELS, 111 EAST WAYNE STREET, SUITE 800, FORT WAYNE, IN, 46802, US

(21) Appl. No.: 11/244,640

(22) Filed: Oct. 6, 2005

(51) Int. Cl. A61B 17/00

(52) U.S. Cl. 606/80

(57) ABSTRACT

A flexible shaft. In exemplary embodiments, the flexible shaft can be used for the transmission of rotary motion and/or curvilinear guidance. In one embodiment, the flexible shaft includes a plurality of shaft segments linked together via at least one flexible elongate member. The shaft segments are spaced along the at least one flexible elongate member. Some of the shaft segments are displaceable relative to the at least one flexible elongate member. Orbiting of the at least one elongate member about a central axis of the shaft induces rotation of the shaft segments about the central axis, thereby effecting rotation of the flexible shaft.

### BACKGROUND

1. Field of the Invention

The present invention relates to flexible shafts and, more particularly, to flexible shafts for the transmission of rotary motion and/or curvilinear guidance.

2. Description of the Related Art

Flexible shafts are useful in many applications, for example, to transmit torque along the shaft, or to guide a device along a path. One exemplary use of flexible shafts is in the medical device field. Flexible shafts may be used for driving a reamer or other instrument, e.g., for driving instruments used to cut bone during orthopedic surgery. In such an application, it is often necessary to cut or ream a curvilinear bore or to compensate for imperfect alignment between the device used to impart rotary motion and a cutting head or other instrument component to which the rotary motion will be imparted. Flexible shafts are also useful, e.g., for providing a curvilinear or straight path over which a tubular structure may be guided, or, if the flexible shaft is cannulated, through which a flexible structure may be guided.

### SUMMARY

The present invention provides a flexible shaft. In exemplary embodiments, the flexible shaft can be used for the transmission of rotary motion and/or curvilinear guidance. In one embodiment, the flexible shaft includes a plurality of shaft segments linked together via at least one flexible elongate member. The shaft segments are spaced along the at least one flexible elongate member. Some of the shaft segments are displaceable relative to the at least one flexible elongate member. Orbiting of the at least one elongate member about a central axis of the shaft induces rotation of the shaft segments about the central axis, thereby effecting rotation of the flexible shaft.

In one form thereof, the present invention provides a flexible shaft including a plurality of discrete shaft segments together defining a first, central axis; and at least one flexible elongate member linking the plurality of shaft segments with at least some of the shaft segments displaceable relative to the elongate member, the elongate member having a second axis spaced from the first axis, wherein orbiting of the at least one elongate member about the first axis causes rotation of each of the plurality of shaft segments about the first axis.

In another form thereof, the present invention provides a flexible shaft including a plurality of discrete shaft segments together defining a first, central axis; and at least one flexible elongate member linking the plurality of shaft segments with at least some of the shaft segments displaceable relative to the elongate member, the elongate member having a second axis spaced from the first axis, the first axis and the second axis together defining a plane of flexure, the at least one flexible elongate member translatable along the second axis to effect flexing of the flexible shaft in the plane of flexure.

### BRIEF DESCRIPTION OF THE DRAWINGS

The above mentioned and other features and objects of this invention, and the manner of attaining them, will become more apparent and the invention itself will be better understood by reference to the following description of embodiments of the invention taken in conjunction with the accompanying drawings, wherein:

FIG. 1A is a perspective view of an exemplary flexible shaft according to the present invention;

FIG. 1B is a perspective view of the flexible shaft of FIG. 1A including spacers located between the shaft segments;

FIG. 1C is a side view of the flexible shaft of FIG. 1B, shown in a curvilinear position including a distal cutter;

FIG. 1D is a sectional view of a shaft segment of the flexible shaft of FIGS. 1A-1C;

FIG. 1E is a perspective view of a flexible shaft according to an alternative embodiment;

FIG. 2A is a top view of an exemplary shaft segment according to another embodiment, the shaft segment including longitudinally oriented cutting blades located on the periphery thereof,

FIG. 2B is a side view of an exemplary flexible shaft having the shaft segment of FIG. 2A, with spaces between adjacent shaft segments;

FIG. 2C is a side view of an exemplary flexible shaft, having substantially no spaces between adjacent shaft segments;

FIG. 3A is a top view of an exemplary shaft segment according to still another embodiment, the shaft segment including inclined cutting blades located around the periphery thereof;

FIG. 3B is a side view of an exemplary flexible shaft including the shaft segment of FIG. 3A;

FIG. 4A is a top view of an exemplary shaft segment according to yet another embodiment, the shaft segment including cutter blades oriented circumferentially around the outer periphery thereof;

FIG. 4B is a side view of an exemplary flexible shaft according to the present invention including the shaft segment of FIG. 4A;

FIG. 5A is an end view of an exemplary shaft segment according to another embodiment;

FIG. 5B is a side view of an exemplary flexible shaft including the shaft segment of FIG. 5A;

FIG. 5C is a side view of the exemplary flexible shaft of FIG. 5B shown in a curvilinear position;

FIG. 5D is a perspective view of an exemplary flexible shaft according to still another embodiment;

FIG. 6A is a side view of an exemplary flexible shaft according to yet another embodiment, the shaft including the shaft segment of FIG. 6C;

FIG. 6B is a side view of the exemplary flexible shaft of FIG. 6A shown in a curvilinear position;

FIG. 6C is an end view of an exemplary shaft segment according to the present invention;

FIG. 7A is a perspective view of the flexible shaft of FIG. 1A housed in a cannulated sleeve for holding the flexible shaft in a desired curvilinear trajectory;

FIG. 7B is a cross-sectional view of the flexible shaft and cannulated sleeve of FIG. 7A;

FIG. 8A is a coronal view of a femur and a curvilinear guide wire placed therein;

FIG. 8B is a coronal view of the femur of FIG. 8A, further illustrating a flexible shaft inserted therein;

FIG. 9A is a perspective view of an exemplary flexible shaft including the shaft segment of FIG. 9B;

FIG. 9B is a cross-sectional view of a shaft segment according to another embodiment; and

FIG. 9C is a side view of the exemplary flexible shaft of FIG. 9A shown in a curvilinear position.

Corresponding reference characters indicate corresponding parts throughout the several views. Although the drawings represent embodiments of the present invention, the drawings are not necessarily to scale and certain features may be exaggerated in order to better illustrate and explain the present invention. The exemplifications set out herein illustrate embodiments of the invention, in several forms, and such exemplifications are not to be construed as limiting the scope of the invention in any manner.

### DETAILED DESCRIPTION

The embodiments disclosed below are not intended to be exhaustive or limit the invention to the precise forms disclosed in the following detailed description. Rather, the embodiments are chosen and described so that others skilled in the art may utilize their teachings.

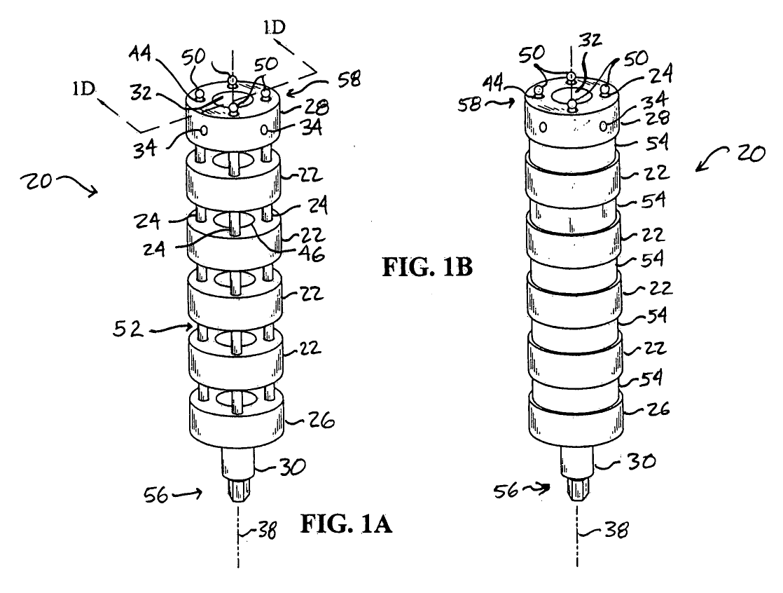

Referring to FIG. 1A, flexible shaft 20 is shown, which includes a plurality of shaft segments 22, at least one flexible elongate member 24, proximal adapter 26, and distal adapter 28. Flexible elongate members 24 link discrete shaft segments 22 together and provide transmission of rotary motion or torque between proximal adapter 26 and distal adapter 28, as described below. Although the embodiment depicted in FIG. 1A includes four flexible elongate members 24, the invention will function with one or more flexible elongate members 24, for example, with only one flexible elongate member 24, shown in FIG. 1E. Flexible elongate member(s) 24 each normally lie along a longitudinal axis which is radially offset from, or eccentric to, longitudinal axis 38 of flexible shaft 20 when flexible shaft 20 is substantially straight, as shown in FIG. 1A.

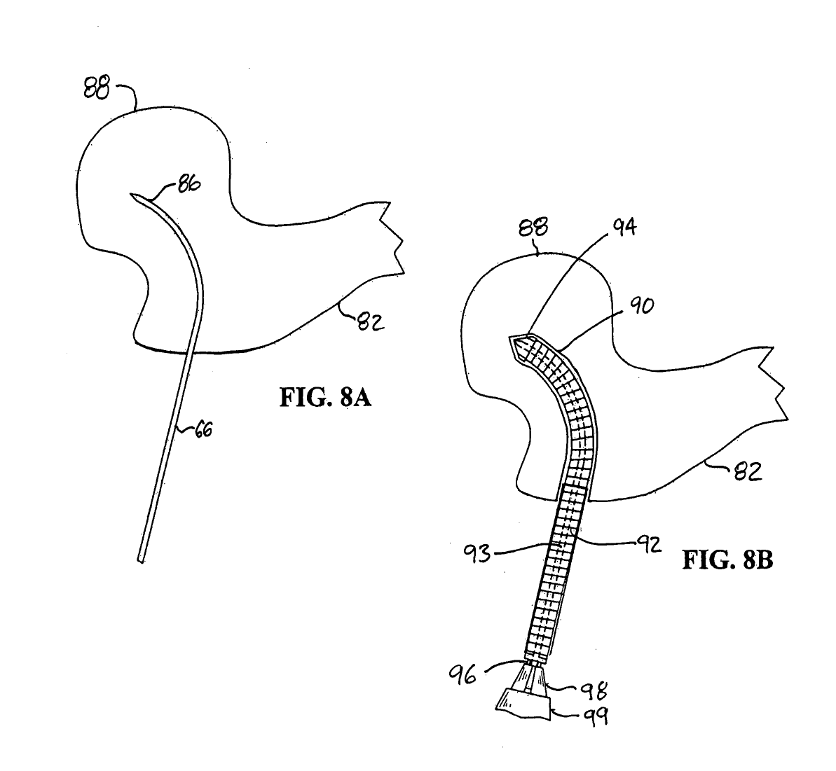

Proximal adapter 26 includes shank 30 for coupling flexible shaft 20 with a chuck or other device such as rotary driver 99, for example, shown in FIG. 8B and discussed below.

Distal adapter 28 may include central bore 32 and fasteners 34, such as set screws, for retaining cutter head 36, for example, shown in FIG. 1C, or another device or medical instrument within central bore 32.

Referring to FIG. 1C, fasteners 34 may be loosened within distal adapter 28 to permit insertion of cutter head shaft 37 of cutter head 36 into central bore 32. Upon insertion of cutter head shaft 37 into central bore 32, fasteners 34 may be tightened to secure cutter head shaft 37 in central bore 32, thereby securing cutter head 36 in flexible shaft 20.

Referring additionally to FIG. 1D, each shaft segment 22 includes at least one bore 44 through which a flexible elongate member 24 passes. Additionally, proximal adapter 26 and distal adapter 28 similarly include at least one bore 44 through which a flexible elongate member 24 passes.

Flexible elongate member 24 is slidable within bores 44. Although illustrated as having circular cross-sectional shapes, flexible elongate member 24 may have a polygonal cross-sectional shape such as rectangular or square. Distal adapter 28 and proximal adapter 26 are retained on flexible elongate members 24 via heads 50 provided at the distal and proximal ends of each flexible elongate member 24. Each head 50 has a dimension larger in diameter than the diameter of bore 44.

As shown in FIG. 1A, head 50 is a sphere which has a diameter larger than that of bore 44. Although illustrated as a sphere, head 50 could take any form which prevents head 50 from substantially entering bore 44 or passing therethrough. Similarly, bore 44 could take any form to accommodate passage of flexible elongate members 24 therethrough, such as a polygonal cross-section. Heads 50 may retain distal adapter 28 and proximal adapter 26 on flexible elongate members 24, and, in turn, shaft segments 22 are retained on flexible elongate members 24. Heads 50 optionally provide tensional strength to flexible shaft 20.

Shaft segments 22 and flexible elongate members 24 may optionally have a friction-fit engagement such that shaft segments 22 may be displaced relative to flexible elongate members 24 when force is applied to shaft segments 22, but shaft segments 22 will remain in place in spaced relationship with respect to one another on flexible elongate members 24 without any force applied to shaft segments 22. Similarly, proximal adapter 26 and distal adapter 28 may have a friction-fit engagement with flexible elongate members 24 and are relatively displaceable with respect to flexible elongate members 24.

Referring still to FIG. 1A, shaft segments 22 may be relatively displaced on flexible elongate members 24 to form voids 52 between adjacent shaft segments. Voids 52 are defined between each proximal end 42 (FIG. 1D) of a shaft segment 22 and each distal end 40 (FIG. 1D) of an adjacent shaft segment 22. The friction-fit engagement of shaft segments 22 and flexible elongate members 24 facilitate the spacing of voids 52 between adjacent shaft segments 22. Without such friction-fit engagement, shaft segments 22 would rest upon one another and voids 52 would not exist.

Referring now to FIG. 1B, flexible shaft 20 optionally may include a plurality of spacers 54 which may encompass voids 52 (FIG. 1A) with each spacer 54 captured between adjacent shaft segments 22. Spacers 54 may shield flexible elongate members 24 from external influences, such as fluids and bone debris, for example. Spacers 54 may be utilized where shaft segments 22 and flexible elongate members 24 have a friction-fit engagement to prevent relative displacement between adjacent shaft segments 22. Alternatively, spacers 54 may be utilized where shaft segments 22 and flexible elongate members 24 do not have a friction-fit engagement wherein spacers 54 function to separate and prevent contact between adjacent shaft segments 22.

The operation of flexible shaft 20 will now be described for the transmission of rotary motion. Upon driving proximal adapter 26 via shank 30 by rotary driver 99, for example, shown in FIG. 8B, flexible elongate members 24 are caused to be orbited around central longitudinal axis 38 of flexible shaft 20. Orbiting of elongate members 24 about central longitudinal axis 38 induces rotation of shaft segments 22 about central longitudinal axis 38.

Rotation of shaft segments 22, in turn, causes flexible shaft 20 to rotate as a unit. Alternatively, one shaft segment 22 other than proximal adapter 26 may be rotated about central longitudinal axis 38 which, in turn, causes orbiting of flexible elongate members 24 about central longitudinal axis 38. Orbiting of flexible elongate members 24 induces rotation of the remainder of shaft segments 22 and proximal and distal adapters 26 and 28 about central longitudinal axis 38.

The operation of flexible shaft 20 will now be described for flexing or placing flexible shaft 20 in a curvilinear position, as shown in FIG. 1C. To flex flexible shaft 20, a user must first stabilize, or fix the proximal adapter 26 in a stationary position. Once the proximal adapter 26 is fixed, tension is applied to one of the flexible elongate members 24. By manner of illustration, in FIG. 1C, the flexible elongate member 24a is pulled a further distance than the remaining flexible elongate members 24b and 24c. Upon pulling or tensioning flexible elongate member 24a in this manner, adjacent shaft segments 22 are pulled together along circumferential segments or adjacent sides thereof by the action of head 50 forcing distal adapter 28 towards adjacent shaft segment 22. Head 50 proximate distal adapter 28 contacts distal adapter 28 and pulls distal adapter 28 towards the first shaft segment 22. This action, in turn, forces the first shaft segment 22 to be pulled towards the next adjacent shaft segment 22. The pulling action subsequently continues to force each shaft segment 22 proximally toward an adjacent shaft segment 22 until proximal adapter 26 is pulled towards an adjacent shaft segment 22. The amount of pulling permitted may be controlled by the material used for spacers 54. Spacers 54, in an exemplary embodiment, may be formed of material that would be susceptible to compression upon tensioning of one flexible elongate member 24, as described above, but remain uncompressed around the remaining circumferential segments. For example, referring to FIG. 1C, spacers 54 are compressed along the left side of flexible shaft 20 but remain uncompressed along the right side of flexible shaft 20.

Referring still to FIG. 1C, upon pulling of flexible elongate member 24a, flexible shaft 20 flexes substantially in a single plane within the drawing sheet of FIG. 1C and is directed to the left as shown. Although not specifically shown in FIG. 1C, it will be recognized that, upon pulling of flexible elongate member 24b, flexible shaft 20 flexes to the right or opposite from flexure due to pulling of member 24a. Pulling of flexible elongate member 24b causes flexure in the same plane as flexure caused by pulling member 24a. Additionally, although not specifically shown in FIG. 1C, it will be recognized that, upon pulling of flexible elongate member 24c, flexible shaft 20 flexes in a plane perpendicular to the flexure plane for flexible elongate members 24a and 24b. When flexible elongate member 24c is pulled, flexible shaft 20 flexes in a direction out of the drawing sheet containing FIG. 1C. In this manner, translating or pulling different flexible elongate members 24 allows a user to bend flexible shaft 20 in a number of different directions. Furthermore, simultaneous activation or pulling of more than one flexible elongate member 24 results in bending movement of flexible shaft 20 out of the perpendicular planes described above. The amount of force required to flex flexible shaft 20 is dependent on the materials chose for shaft segments 22, flexible elongate members 24, and spacers 54, if used.

Referring now to FIG. 1D, shaft segment 22 may have a cylindrical cross-sectional shape including substantially parallel distal end 40 and proximal end 42 with central bore 46 substantially coaxial with central longitudinal axis 38 of flexible shaft 20. Shaft segment 22 may also include outer periphery surface 62. Shaft segment 22 optionally may have a polygonal cross-sectional shape. Shaft segments 22 optionally may be rigidly constructed, for example, from stainless steel. A rigid construction of shaft segment 22 essentially means that upon contact with an adjacent shaft segment 22, neither shaft segment 22 will deform, but instead will retain its original shape. Shaft segments 22 may also be formed of shape-memory material which deforms under pressure, e.g., compression, and returns to its original shape once the pressure is released. Such a construction may permit greater flexibility of flexible shaft 20 when adjacent shaft segments 22 contact each other. Flexible elongate members 24 may be mono- or multi-filament braided or nonbraided cable made of various materials including, for example, stainless steel, cobalt chrome alloy, shape memory alloys (e.g., nitinol), polymeric material, or woven material.

Materials suitable for shaft segment 22, flexible elongate members 24, and spacers 54 may include any material acceptable for surgical instrumentation use. The material would be selected based on desired shaft behavior and functional requirements. For shaft segment 22, for a multiple-use, high-wear or high-torque application, a metal might be used. Additionally, for a single-use or low-strength application of shaft segment 22, a polymeric material may be used for the potential purpose of cost savings or economy of manufacturing. For flexible elongate members 24, an exemplary construction would include a monofilament wire of a fairly elastic metallic material to enhance strength and flexibility. A multifilament wire may also be used for flexible elongate members 24 to offer a wider variety of flexibility and strength. Furthermore, flexible elongate members 24 may be constructed of a monofilament, i.e., a polymeric rod, or multifilament polymeric constructions, i.e., woven or braided textiles. For spacers 54, a highly elastic polymer, e.g., an elastomer, may be used, or, alternatively, a metallic material or other polymers may be used for possible advantages in manufacturing or strength.

The transmission of torque through flexible shaft 20 is dependent on several critical factors. The location of flexible elongate members 24 in shaft segments 22 relative to central longitudinal axis 38 determines the torque transmission capabilities. For example, if flexible elongate member 24 were located coaxial with central longitudinal axis 38, little or no torque transmission would be available through flexible shaft 20. If flexible elongate members 24 are spaced a radial distance from central longitudinal axis 38, the torque transmission capabilities of flexible shaft 20 is enhanced if flexible shaft 20 is driven from proximal adapter 26.

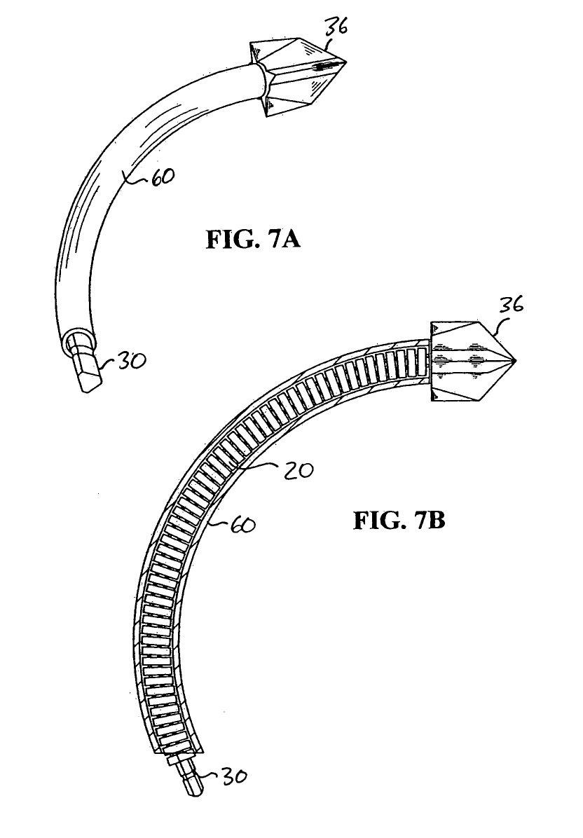

In one example, a curvilinear guide, such as cannulated curvilinear tube 60 shown in FIGS. 7A-7B, may be slid around outer periphery surfaces 62 (FIGS. 1C-1D) of shaft segments 22 in order to guide flexible shaft 20 into the shape of tube 60. As flexible shaft 20 enters tube 60, shaft segments 22 are displaced in order to conform to the curvilinear shape of tube 60. In an alternative embodiment, inner wall 64 (FIG. 1D) of central bore 46 of shaft segment 22 may provide passage therethrough of a curvilinear guide, such as guide wire 66 shown in FIG. 8A, as described below. As flexible shaft 20 receives guide wire 66, flexible shaft 20 is flexed to conform to the curvilinear shape of guide wire 66.

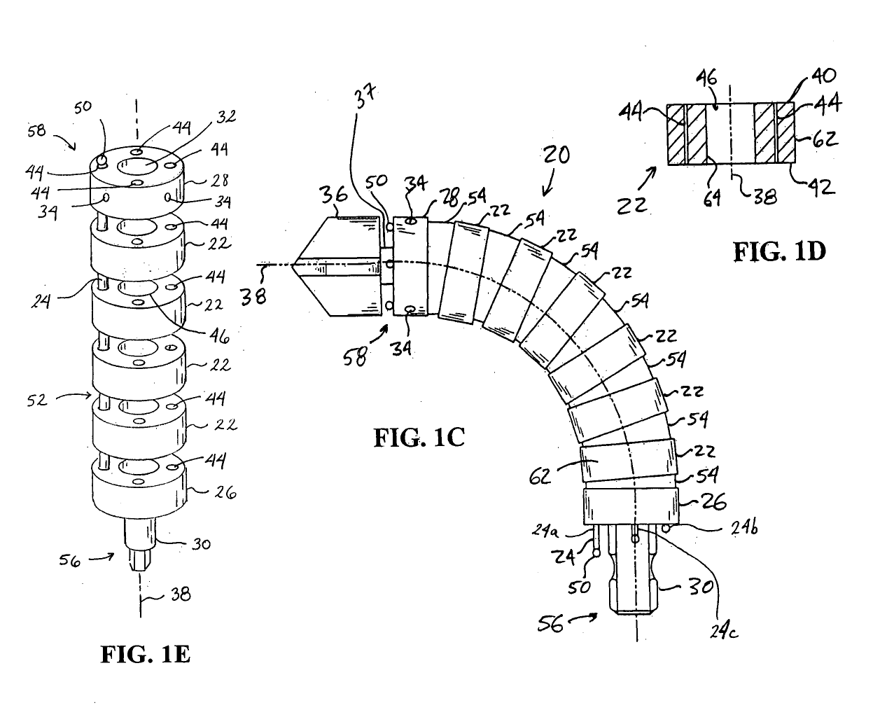

In one alternative embodiment, shown in FIGS. 2A through 2C, flexible shaft 100 is shown which, except as described below, is substantially similar in structure and operation to flexible shaft 20 (FIGS. 1A-1C) described above. Shaft 100 includes shaft segments 102, bores 120, and heads 122 to retain shaft segments 102 on flexible elongate members 104. Flexible shaft 100 may include blades 106 disposed around the outer circumference thereof to form, for example, a reaming instrument. Blades 106 may include cutting edges substantially parallel to longitudinal axis 110 of flexible shaft 100. Each blade 106 is defined between face 112 and land 114. Flutes 116 divide adjacent blades 106. Face 112 may be oriented such that, relative to the direction of rotation, blade 106 is angled forward of a line extending from longitudinal axis 110 to the point where face 112 meets flute 116. Each shaft segment 102 may have central bore 118, thereby allowing guide wire 66 (FIGS. 8A-8B) or flexible shaft 20 (FIGS. 1A-1C) to be inserted through flexible shaft 100. In the latter manner, flexible shaft 100 may be oriented over flexible shaft 20 to provide flexible shaft 20 with reaming capabilities.

In another alternative embodiment, shown in FIG. 2C, reamer 140 is shown which, except as described below, is substantially similar in structure and operation to flexible shaft 100 (FIGS. 2A-2B) described above. Reamer 140 includes shaft segments 146, flexible elongate members 144, and heads 142. Because of the close proximity of adjacent shaft segments 146, reamer 140 may have substantially less ability to flex to a particular configuration. In a similar manner to flexible shaft 100, flexible shaft 20 may be inserted through reamer 140 to provide greater flexibility to reamer 140. Reamer 140 may further include reamer blades 148 which are similar to blades 106 (FIG. 2A), as described above.

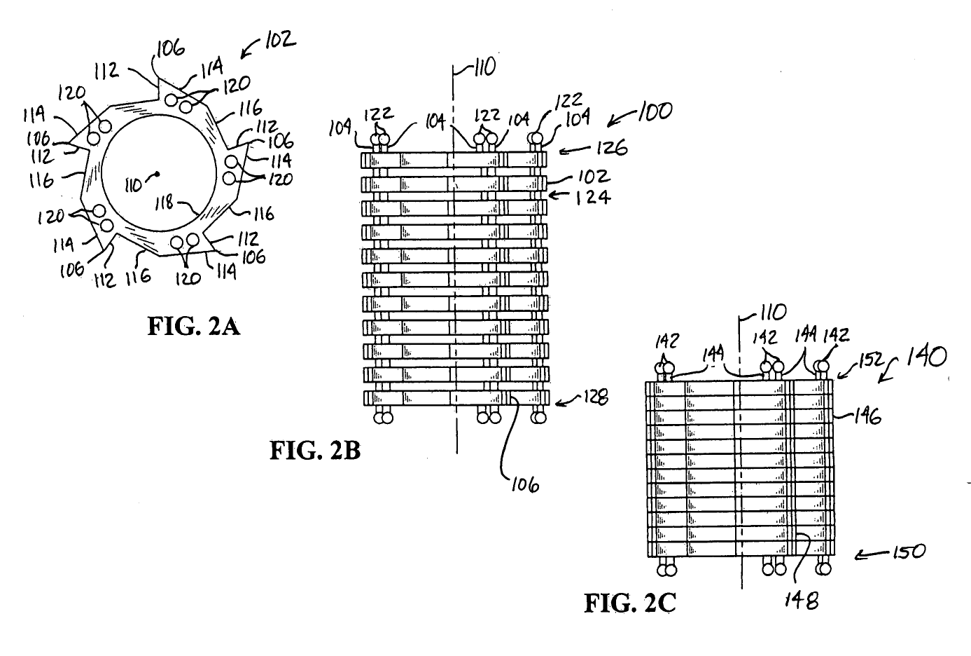

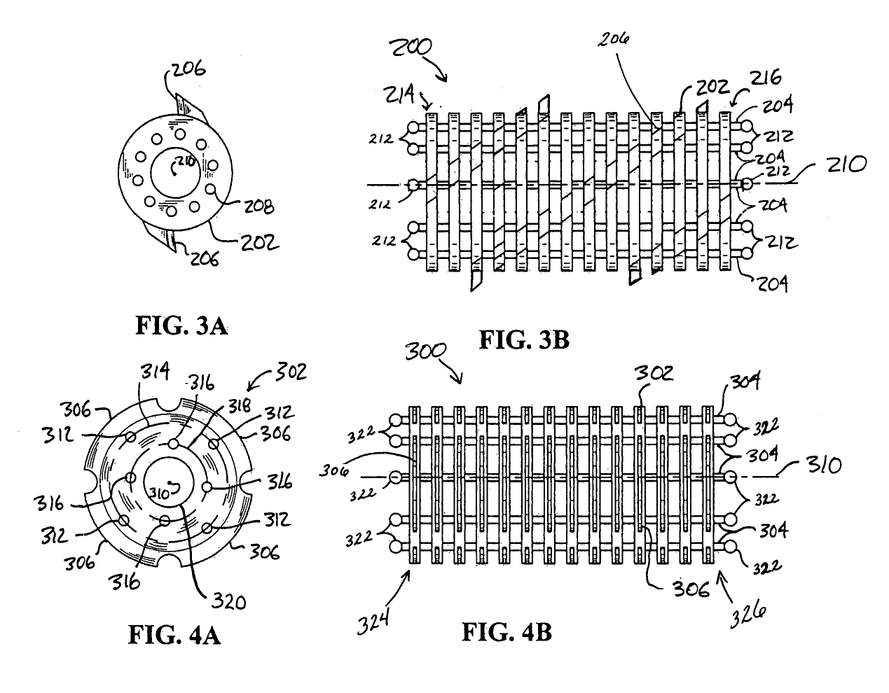

Referring now to FIGS. 3A, 3B, 4A, and 4B, alternative embodiments of the flexible shaft of the present invention are shown as flexible shaft 200 (FIGS. 3A-3B) and flexible shaft 300 (FIGS. 4A-4B). Flexible shafts 200 and 300 are shown which, except as described below, are substantially similar in structure and operation to flexible shaft 100 (FIGS. 2A-2B) described above. Referring to FIGS. 3A and 3B, flexible shaft 200 includes shaft segments 202, bores 208, and heads 212 to retain shaft segments 202 on flexible elongate members 204. Blades 206 of segments 202 of shaft 200 protrude from an outer circumference of each shaft segment 202 and are oriented oblique relative to longitudinal axis 210 of flexible shaft 200. Referring to FIGS. 4A and 4B, flexible shaft 300 includes shaft segments 302, bores 312, 316, and heads 322 to retain shaft segments 302 on flexible elongate members 304. Blades 306 of segments 302 of shaft 300 extend circumferentially around segments of the outside circumference of each shaft segment 302 and extend in a plane substantially perpendicular to longitudinal axis 310 of flexible shaft 300.

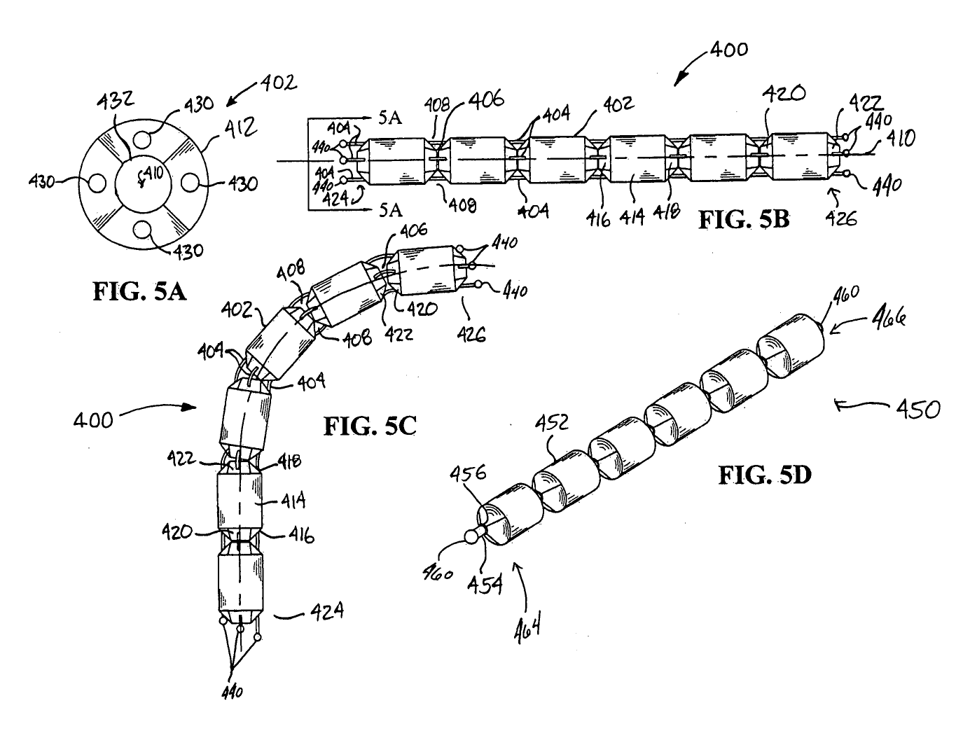

Referring now to FIGS. 5A through 5C, a further alternative embodiment flexible shaft 400 is shown which, except as described below, is substantially similar in structure and operation to flexible shaft 20 (FIGS. 1A-1C) described above. Flexible shaft 400 includes shaft segments 402, bores 430, and heads 440 to retain shaft segments 402 on flexible elongate members 404. Each shaft segment 402 includes distal sloped surface 420 and proximal sloped surface 422 to define angled void 408 advantageously facilitating the flexing of flexible shaft 400. This arrangement accommodates flexing of flexible shaft 400, i.e., angled faces 420 and 422 permit greater localized proximity of pairs of adjacent shaft segments 402 during flexing of shaft 400.

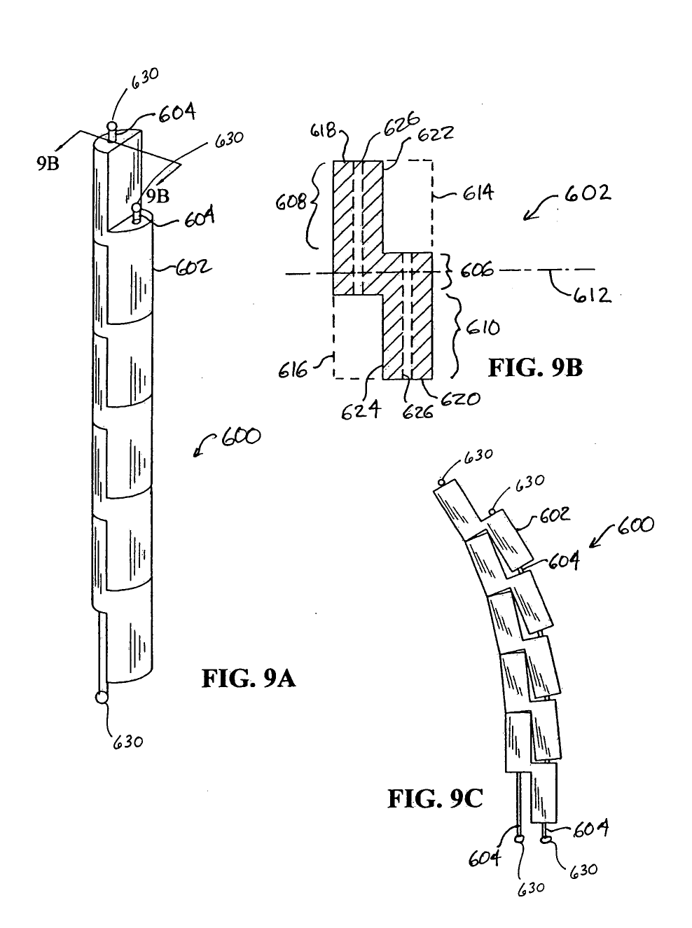

Referring now to FIG. 5D, a still further embodiment flexible shaft 450 is shown which, except as described below, is substantially similar in structure and operation to flexible shaft 20 (FIGS. 1A-1C) described above. Flexible shaft 450 includes shaft segments 452, flexible elongate member 454, and heads 460. Shaft segments 452 of flexible shaft 450 may each include bore 456 and a single flexible elongate member 454 may be used to link shaft segments 452 together. Flexible shaft 450 may be injection molded with shaft segments 452 and flexible elongate member 454 integrally formed. In an exemplary embodiment, flexible shaft 450 may be used in conjunction with a curvilinear guide, such as cannulated curvilinear tube 60 shown in FIGS. 7A-7B, to facilitate flexing flexible shaft 450. In another embodiment, flexible shaft 450 may be used with a curvilinear guide and function as a push rod to impart axial loads. In one alternative embodiment, shaft segments 602 (FIGS. 9A-9C) may be used in flexible shaft 450 to give flexible shaft 450 the ability to transit torque through flexible shaft 450.

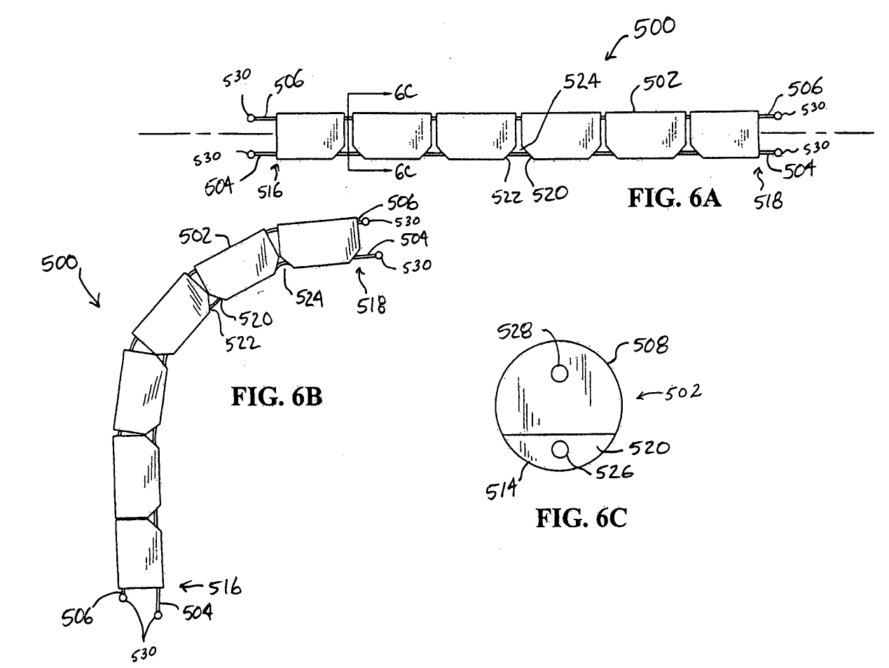

Referring now to FIGS. 6A through 6C, alternative embodiment flexible shaft 500 is shown which, except as described below, is substantially similar in structure and operation to flexible shaft 400 (FIGS. 5A-5C) described above. Flexible shaft 500 includes shaft segments 502, bores 526, 528, and heads 530 to retain shaft segments 502 on flexible elongate members 504, 506. Distal sloped surface 520 and proximal sloped surface 522 extend around portion 514 of the circumference of each shaft segment 502. In this manner, flexing of flexible shaft 500 in a single direction/plane is facilitated or guided. The absence of sloped surfaces 520 and 522 around the entire circumference of shaft segments 502, including circumferential segment 508, does not prevent flexing in any other direction or plane except the plane and direction shown in FIG. 6B, but instead, the presence of sloped surfaces 520 and 522 merely facilitates flexing in the plane and direction as shown in FIG. 6B.

Referring now to FIGS. 9A through 9C, alternative embodiment flexible shaft 600 is shown which, except as described below, is substantially similar in structure and operation to flexible shaft 20 (FIGS. 1A-1C) described above. Flexible shaft 600 includes shaft segments 602, bores 626, and heads 630 to retain shaft segments 602 on flexible elongate members 604. Shaft segments 602 may include central portion 606, distal flexible elongate portion 608, and proximal flexible elongate portion 610. Shaft segments 602 may be positioned on flexible elongate members 604 so that proximal flexible elongate portion 610 of shaft segment 602 overlaps distal flexible elongate portion 608 of adjacent shaft segment 602, as shown in FIGS. 9A and 9C. In this manner, distal interior surface 622 and proximal interior surface 624 of adjacent shaft segments 602 contact to help transmit torsional forces through adjacent shaft segments 602. The shape of shaft segments 602 facilitates the transmission of torque through flexible shaft 600 due to the overlapping configuration of adjacent shaft segments 602.

Although flexible shafts have numerous applications, one application is for rotationally driving a medical instrument. For example, minimally invasive surgical methods for reducing femoral fractures may utilize a flexible shaft to provide curvilinear boring of a femoral head. Referring to FIGS. 8A and 8B, guide wire 66 may be placed through a minimally invasive surgical incision to facilitate reaming curvilinear bore 90 into femoral head 88 of femur 82. Methods and apparatuses for forming bore 90 are disclosed and discussed in detail in the following references: U.S. Pat. No. 6,447,514, entitled “Polymer Filled Hip Fracture Fixation Device,” issued Sep. 10, 2002; U.S. patent application Ser. No. 10/155,683, entitled “Method and Apparatus for Reducing Femoral Fractures,” filed May 23, 2002; U.S. patent application Ser. No. 10/266,319, entitled “Telescoping Reamer,” filed Oct. 8, 2002; U.S. patent application Ser. No. 10/358,009, entitled “Method and Apparatus for Reducing Femoral Fractures,” filed Feb. 4, 2003; U.S. patent application Ser. No. 11/061,898, entitled “Method and Apparatus for Reducing Femoral Fractures,” filed Feb. 18, 2005; U.S. Provisional Patent Application Ser. No. 60/621,487, entitled “Method and Apparatus for Reducing Femoral Fractures,” filed Oct. 22, 2004; and U.S. Provisional Patent Application Ser. No. 60/654,481, entitled, “Method and Apparatus for Reducing Femoral Fractures,” filed Feb. 18, 2005, the disclosures of which are hereby explicitly incorporated by reference herein.

In operation, guide wire 66 includes curvilinear portion 86 which is driven into femoral head 88 and acts as a guide for proper placement of curvilinear bore 90, shown in FIG. 8B. Flexible shaft 92, having distal cutter 94 and proximal adapter 96, can be coupled at proximal adapter 96 to chuck 98 of driver 99. Flexible shaft 92 may be substantially similar in structure and operation to flexible shaft 20 (FIGS. 1A-1C) or any other of the flexible shafts described above. Central bore 93, which extends through flexible shaft 92 and distal cutter 94, may receive guide wire 66 and, as flexible shaft 92 is received onto guide wire 66, flexible shaft 92 flexes to the curvilinear shape of guide wire 66 while bore 90 is created. Therefore, rotary driving of distal cutter 94 and movement of flexible shaft 92 along guide wire 66 provides guided cutting of curvilinear bore 90 in femoral head 88 of femur 82.

While this invention has been described as having preferred designs, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. A flexible shaft, comprising:
a plurality of discrete shaft segments together defining a first, central axis; and
at least one flexible elongate member linking said plurality of shaft segments with at least some of said shaft segments displaceable relative to said elongate member, said elongate member having a second axis spaced from said first axis, wherein orbiting of said at least one elongate member about said first axis causes rotation of each of said plurality of shaft segments about said first axis.

2. The flexible shaft of claim 1, further comprising at least two said flexible elongate members at least one of which is translatable to effect pulling of said shaft segments and flexing of the flexible shaft.

3. The flexible shaft of claim 2, wherein the flexible shaft is flexible in a single plane of flexure defined by said first axis and said second axis upon translation of said one flexible elongate member.

4. The flexible shaft of claim 1, wherein the flexible shaft includes a proximal segment, said proximal segment including a coupling structure.

5. The flexible shaft of claim 1, further comprising a plurality of spacers respectively disposed between adjacent pairs of said discrete shaft segments.

6. The flexible shaft of claim 1, wherein the flexible shaft includes a distal segment, said distal segment including a medical instrument.

7. The flexible shaft of claim 1, wherein at least some of said discrete shaft segments include at least one cutting surface.

8. The flexible shaft of claim 1, wherein said at least one flexible elongate member includes first and second head portions on opposite ends thereof, said head portions retaining said shaft segments on said at least one flexible elongate member.

9. The flexible shaft of claim 1, further comprising a plurality of said flexible elongate members, at least some of said plurality of flexible elongate members translatable to effect pulling of said shaft segments and flexing of the flexible shaft.

10. The flexible shaft of claim 1, wherein at least some of said discrete shaft segments are frictionally fitted with respect to said at least one flexible elongate member.

11. The flexible shaft of claim 1, wherein said shaft segments may be relatively displaced on said flexible elongate members to form voids between adjacent shaft segments.

12. The flexible shaft of claim 1, wherein said shaft segments are rigidly constructed.

13. The flexible shaft of claim 1, wherein said shaft segments are formed of shape-memory material which deforms under pressure, and returns to its original shape once the pressure is released.

14. A flexible shaft, comprising:
a plurality of discrete shaft segments together defining a first, central axis; and
at least one flexible elongate member linking said plurality of shaft segments with at least some of said shaft segments displaceable relative to said elongate member, said elongate member having a second axis spaced from said first axis, said first axis and said second axis together defining a plane of flexure, said at least one flexible elongate member translatable along said second axis to effect flexing of the flexible shaft in said plane of flexure.

15. The flexible shaft of claim 14, wherein the flexible shaft includes a proximal segment, said proximal segment including a coupling structure.

16. The flexible shaft of claim 14, further comprising a plurality of spacers respectively disposed between adjacent pairs of said discrete shaft segments.

17. The flexible shaft of claim 14, wherein the flexible shaft includes a distal segment, said distal segment including a medical instrument.

18. The flexible shaft of claim 14, wherein at least some of said discrete shaft segments include at least one cutting surface.

19. The flexible shaft of claim 14, wherein said at least one flexible elongate member includes first and second head portions on opposite ends thereof, said head portions retaining said shaft segments on said at least one flexible elongate member.

20. The flexible shaft of claim 14, further comprising a plurality of said flexible elongate members, at least some of said plurality of flexible elongate members translatable to effect pulling of said shaft segments and flexing of the flexible shaft in a plurality of flexure planes.

21. The flexible shaft of claim 14, wherein at least some of said discrete shaft segments are frictionally fitted with respect to said at least one flexible elongate member.

22. The flexible shaft of claim 14, wherein orbiting of said at least one flexible elongate member about said first axis causes rotation of each of said plurality of shaft segments about said first axis.

23. The flexible shaft of claim 14, wherein rotation of one of said plurality of shaft segments about said first axis causes orbiting of said at least one flexible elongate member about said first axis, wherein orbiting of said at least one flexible elongate member about said first axis causes rotation of the remainder of said plurality of shaft segments about said first axis.
